(19) United States
(12) Patent Application Publication
(10) Pub. No.: US 2004/0135104 A1
(43) Pub. Date: Jul. 15, 2004

(54) System for the microbiological disinfection of ventilation and air-conditioning ducts

(76) Inventor: Maria Teresa Vilarasau Alegre, Sant Antoni De Viamajor (ES)

Correspondence Address: Maria Parrish Tungol, Suite 600, 1800 Diagonal Road, Alexandria, VA, 22314, US

(21) Appl. No.: 10/719,945

(22) Filed: Nov. 21, 2003

Related U.S. Application Data
(62) Division of application No. 09/582,804, filed on Jun. 30, 2000, now Pat. No. 6,653,647.
Application No. 09/582,804 is a 371 of international application No. PCT/ES98/00296, filed on Nov. 3, 1998.

(51) Int. Cl. A61L 9/20

(52) U.S. Cl. 250/504 R

(57) ABSTRACT

A mobile device (3) is adapted to be inserted inside a ventilation or air-conditioning duct (2) and moved inside in order to disinfect the duct. This device comprises an axle (31) on which ultraviolet ray lamps (34) are assembled, end discs (32 and 33) provided with wheels (35 and 36) on the edge, and can further comprise traction and retention cables (38 and 39).

This application is a divisional of application Ser. No. 09/582,804 filed Jun. 3, 2000 which is the National Stage of International Application No. PCT/ES9800296 filed Nov. 3, 1998.

The system of this invention consists of a static microbiological disinfection device by ultraviolet rays, which is permanently assembled at the air inlet or inlets in the duct, so that the micro-organisms contained in the air that is projected inside the circuit by the corresponding ventilation or cooling equipment, are eliminated, plus a mobile microbiological disinfection device by ultraviolet rays, which is placed inside the duct to be cleaned, and which is moved along the length of the duct eliminating any possible micro-organisms which could be inside.

### BACKGROUND TO THE INVENTION

Ventilation and air conditioning ducts are currently cleaned by combining the application of pressurised air on the inside walls with suction from outside the duct; by applying pressurised air, adhered dust particles are separated from the walls, and with suction, these particles are removed from the duct.

Although these mechanical cleaning devices provide good surface cleaning of dust particles and dirt stuck to the duct, they are not sufficient to microbiologically clean. However, it is known that micro-organisms multiply in these ducts, causing many respiratory disorders.

This microbiological cleaning method is attempted by spraying the inside of the duct with disinfectant, that can cause discomfort to certain people, or be harmful to health. Owing to its oxidising power, it can damage the, ducts.

### DESCRIPTION OF THE INVENTION

To solve these problems, the system that is the purpose of the invention has been designed, aimed at microbiologically cleaning the duct by emitting short-wave ultraviolet rays.

In accordance with the invention, this system is composed of at least one static device which is assembled inside the duct, near the air inlet connected to the corresponding cooling or ventilation equipment.

This static device is composed of a set of short-wave ultraviolet lamps, the purpose of which is to disinfect the air flow entering the duct. It has been planned that these lamps are connected to the corresponding cooling or ventilation equipment, and are kept on while said equipment is operating. This ensures that the incoming air does not contain micro-organisms and is not a continuous source of bacteria, fungus or other simple forms of life inside the duct.

Bearing in mind that these micro-organisms can enter the duct through the ventilation outlets, when there is no forced circulation of air inside, this system is complemented by a mobile device, inserted inside the duct from time to time, and moved inside, thereby fully disinfecting the duct.

According to the invention, this mobile device will be composed of a central axle, around which a series of short-wave ultraviolet ray lamps are assembled, and of two end discs with wheels on the edge forming the support means of the device on the inside surface of the duct.

The lamps of the mobile device are placed parallel to themselves and also to the central axle, emitting ultraviolet radiation which covers an area of 360° around the axle.

This mobile device is provided with the corresponding tubing for electric power of the lamps and two resistant cables which are fixed to the opposite ends of the device.

In order to disinfect any part of the duct, the mobile device simply has to be positioned inside, removing the electric input tubing and one of the resistant cables through one of the ventilation openings, and the remaining cable through another of the ventilation outlets.

One of these cables will be used to pull the mobile device causing it to move inside the duct, while the other cable will be used as a retainer, to prevent the device from suddenly accelerating when circulating through a slanted part or through vertical parts of the duct.

In order to fully disinfect the duct by means of the mobile device, the characteristics of the duct should first be observed, such as: type of material of which it is composed, the reflectance index, section, surface roughness, etc.

The radiation applied in each case can be achieved by varying the forward speed of the mobile device, and maintaining the power of the lamps constant, or varying the power of the lamps and maintaining the forward speed of the device constant.

In both cases, in order to set the forward speed of the device, at least one of the cables fixed to the mobile device is adjusted.

### DESCRIPTION OF THE DRAWINGS

FIG. 1 shows a plan section of an air conditioning duct, with the disinfection devices included in the system that it is the object of this invention drawn inside.

FIG. 2 shows a perspective view of the static disinfection device.

FIG. 3 shows a plan detail of the mobile disinfection device.

FIG. 4 shows a cross section of the same mobile device.

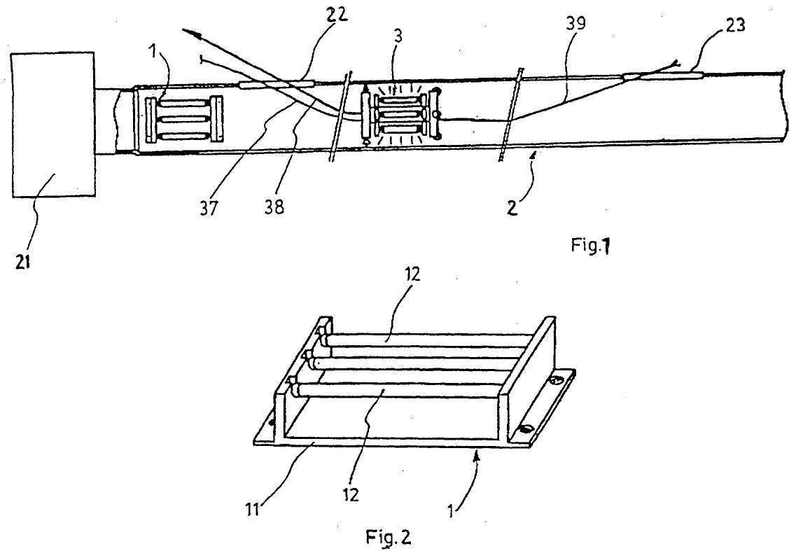

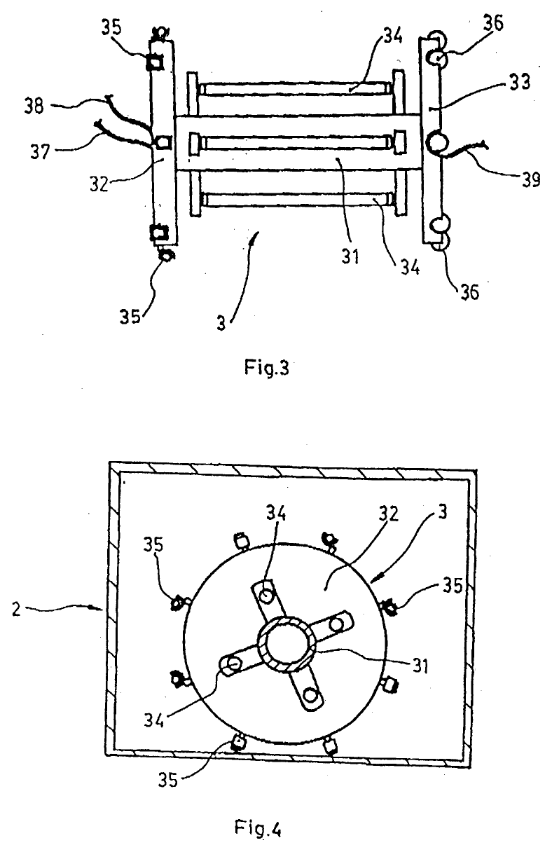

### PREFERENTIAL MANUFACTURE OF THE INVENTION

As can be observed in the drawings, the microbiological disinfection system that is the object of the invention, consists of a static device (1) to be fixed inside the duct (2) to disinfect the incoming air flow from cooling equipment (21) and a mobile device (3) which will be periodically used, after mechanically cleaning the ducts, to fully disinfect the duct (2).

The static device (1) consists of a support (11) provided with a series of lamp holders to assemble a set of lamps (12) which emit short-wave ultraviolet rays. This device (1) is permanently fixed inside the duct (2) near the air inlet coming from the equipment (21), and electrically connected to the equipment (21) itself, so that the lamps (12) are on when said equipment (21) is also on. In this way, the incoming air flow crosses the radiation area, removing the micro-organisms contained inside.

Bearing in mind that micro-organisms can get inside the duct (2) through the ventilation outlets (22 and 23) when the equipment (21) is off, it is necessary to periodically disinfect the whole duct (2).

This operation will be carried out by means of the mobile device (3), preferably having cleaned the inside of the duct by any known method.

The mobile device consists of an axle (31) with end discs (32 and 33). The axle (31) has lamp holders on which ultraviolet ray lamps (34) are assembled and positioned around the axle (31) and parallel to it.

The end disc (32) positioned in the front part of the device in the forward direction, has wheels (35) around its edge which are parallel to the axle (31), while the end disc (33) has wheels (36) around its edge, which are also parallel to the axle (31).

This device (3) also has tubing (37) for the electric input of the lamps (34) and two resistant cables (38 and 39) fixed on the front and back ends of the axle (31).

As can be observed in the drawing (1), in order to clean the duct (2) by means of a mobile device (3), this is inserted inside the conduct (2) so that the axle (31) is directed longitudinally and the cables (38 and 39) project outside through ventilation openings (22 and 23) respectively.

By means of cable (38), the device (3) is manually or mechanically pulled causing it to advance inside the duct (2), and it is slightly retained by cable (39) to ensure that the device (3) moves in the correct position with device axles-duct in parallel, and at a fairly constant speed, even in slanted or vertical parts of the duct (2).

Owing to the lay-out of the lamps (34) around the axle (31), the movement of the device (3) ensures that all the inside surface of the duct (2) is subject to ultraviolet radiation of sufficient intensity and duration to achieve total microbiological disinfection.

In order to control the forward speed of the device (3), it is planned that at least one of the cables (38, 39) is adjusted.

What is claimed is:

1. A mobile device (3) for disinfecting a ventilation or air-conditioning duct wherein the device is adapted to be inserted inside the duct, the device comprising an axle (31) operably connected to a front end disc (32) and to a rear end disk (33) wherein axle (31) is also operably connected to at least one ultraviolet ray lamp (34) positioned around axle (31).

2. The device of claim 1 wherein ultraviolet ray lamp (34) is parallel to the axle.

3. The device of claim 1 wherein the axle is operably connected to a plurality of ultraviolet ray lamps.

4. The device of claim 1 wherein the axle is operably connected to a plurality of ultraviolet ray lamps parallel to the axle (31) wherein the lamps cover a radiation area of 360° in a perpendicular plane to the axle.

5. The device of claim 1 further comprising at least one revolving means attached to an edge of front end disc (32) and at least one revolving means attached to an edge of rear end disk (33).

6. The device of claim 5 wherein the revolving means are wheels (35), (36) positioned parallel to axle (31).

7. The device of claim 1 further comprising tubing (37) for electric input to lamps (34).

8. The device of claim 1 further comprising means for positioning the device at a desired location inside the duct.

9. The device of claim 8 wherein the means for positioning the device comprises a cable (38) affixed to front end of axle (31).

10. The device of claim 8 wherein the means for positioning the device comprise a cable (38) affixed to front end of axle (31) and a cable (39) fixed to back end of the axle (31).

11. A mobile device (3) for microbiologically disinfecting the interior of a ventilation or air-conditioning duct, the device comprising an axle (31) operably connected to a front end disc (32) and to a rear end disk (33) wherein the axle (31) is operably connected to a plurality of ultraviolet ray lamps (34) positioned around and parallel to axle (31), front end disc (32) having a plurality of wheels (35) positioned on an edge of disc (32) and parallel to axle (31), rear end disk (33) having a plurality of wheels (36) positioned on an edge of disc (33) and parallel to axle (31), cable (38) affixed to front end of axle (31) and cable (39) fixed to back end of the axle (31).
